(19) United States
(12) Patent Application Publication
(10) Pub. No.: US 2014/0206943 A1
(43) Pub. Date: Jul. 24, 2014

(54) LIGHT GUIDE FOR ASSISTING INTUBATION

(71) Applicant: NATIONAL CENTRAL UNIVERSITY, Taoyuan County (TW)

(72) Inventors: Ching-Cheng SUN, Taoyuan County (TW); Jhih-You CAI, TAOYUAN COUNTY (TW); Fu-Jen CHEN, Taichung City (TW)

(73) Assignee: NATIONAL CENTRAL UNIVERSITY, Taoyuan County (TW)

(21) Appl. No.: 13/745,370

(22) Filed: Jan. 18, 2013

(51) Int. Cl. A61M 16/04; A61B 1/06; A61B 1/07

(52) U.S. Cl. CPC A61M 16/0488; A61B 1/07; A61B 1/0684
USPC 600/249

(57) ABSTRACT

A light guide for assisting intubation can be used to assist the intubation, and includes a catheter and a light emitting element. The emitted light penetrates the surface of the animal's body out from the interior of the animal so as to recognize the location of the catheter for facilitating the subsequent intubation.

### BACKGROUND OF THE INVENTION

1. Field of the Invention

The present invention relates to provide a light guide for assisting intubation, and particularly to a light guide which uses a light emitting element to emit a light from a front end of a catheter to penetrate through the surface of animal's body from the interior of the animal so as to recognize the location of the catheter for facilitating the subsequent intubation.

2. Description of Related Art

The traditional medical intubation is to use a laryngoscope inserting into the mouth of the patient. A front end of the laryngoscope has a blade. When the patient's tongue is pushed away and the blade is placed to the junction of the base of the tongue and epiglottis, the medical personnel can see the vocal cord then the endotracheal tube inserted. In this way when in emergency rescue or general intubation, the patient will feel uncomfortable as hard to see the patient's vocal cord, which may cause incorrect positioning of intubation. Many existing devices attempt to solve the above problems by using the similar endoscopic photography device.

However, the disadvantage of endoscopic photography device is that the medical personnel must look at the images captured by the photographing device to determine the path of intubation. It is not easy to precisely and directly operate due to the screen of endoscopic photography device a lot of time will be masked by sputum or blood from the patient. Furthermore, the cost of such devices is expensive.

For this reason, in order to avoid any regret caused by the incorrect intubation and offer more convenience in use for physicians, the present invention has developed a light guide for assisting intubation through the guidance of the emitted light to offer physicians the right intubation position, reducing the risk of intubation.

### SUMMARY OF THE INVENTION

A main purpose of this invention is to provide a light guide for assisting intubation so that the user can quickly and precisely judge the location of a catheter by the assistance of the emitted light.

In order to achieve the above and other objectives, the light guide for assisting intubation according to the invention includes a catheter and a light emitting element. The catheter is made of material having plasticity. The light emitting element emits a light which can penetrate through the surface of an animal's body and mounted at one end of the catheter. When the end of the catheter having no the light emitting element inserts in the animal's body, the light from the light emitting element penetrates through the surface of the animal's body to indicate the location of that end of the catheter.

Preferably, the light emitting element is a light source having directivity to increase the convenience in recognizing the location.

Preferably, the light emitting element is a light emitting diode.

Preferably, the light from the light emitting element is the visible light having wavelength of 590 nm-760 nm.

Preferably, the light emitting element is mounted to the catheter in a detachable manner for the purpose of replacement or cleaning.

In another embodiment, the light guide for assisting intubation according to the invention includes a catheter and a light emitting element. The catheter is made of material having plasticity and light transmissibility. The light emitting element emits a light which can penetrate through the animal's body and mounted at one end of the catheter. When the end of the catheter having light emitting element inserts in the animal's body, the light from the light emitting element travels through the catheter, goes out of other end of the catheter and then penetrates through the animal's body to indicate the location of that end of the catheter.

The light guide of claim 6, wherein the catheter is made of optical fiber.

The light guide of claim 6, wherein the end of the catheter where the light emitting element is provided with has a light receiving element to receive the light from the light emitting element for guiding the light into the catheter. Thereby, the location of the front end of the catheter can be observed from the animal's body, facilitating the implementation of intubation.

Preferably, the catheter is made of optical fiber.

Preferably, the end of the catheter where the light emitting element is provided with has a light receiving element to gather the light emitted from the light emitting element so as to guide the light into the catheter.

Preferably, the end of the catheter where the light emitting element is not provided with has a light scattering element to scatter the light.

In light of the above, the light guide of the invention offers the following advantages.

(1) The light emitted from the light emitting element mounted at one end of the catheter helps the medical personnel to judge the current location of the catheter in real time, avoiding any incorrect positioning of the catheter.

(2) The light emitted from the light emitting element mounted at one end of the catheter directly transmits through the animal's body so that the medical personnel can visually judge the current location of the catheter, in order to sufficiently take advantage of the golden time for rescuing patients.

(3) The light guide of the invention can be detachably mounted to the intubation for general rescue. The catheter can be inserted first. After the location where the intubation is operated is confirmed, the catheter is inserted along the catheter to precisely reach the correct location, allowing the medical personnel quickly and precisely complete the intubation.

### BRIEF DESCRIPTION OF THE DRAWINGS

FIG. 1 is a schematic view of a light guide for assisting intubation according to a first embodiment of the invention.

FIG. 2 is a schematic view of a light guide for assisting intubation according to a second embodiment of the invention.

FIG. 3 is a schematic view of a light receiving element and a light scattering element of a light guide for assisting intubation according to the second embodiment of the invention.

FIG. 4 is a schematic view of intubation assisting by means of the light guide according to one embodiment of the invention.

### DETAILED DESCRIPTION OF THE PREFERRED EMBODIMENTS

The aforementioned illustrations and following detailed descriptions are exemplary for the purpose of further explaining the scope of the present invention. Other objectives and advantages related to the present invention will be illustrated in the subsequent descriptions and appended tables.

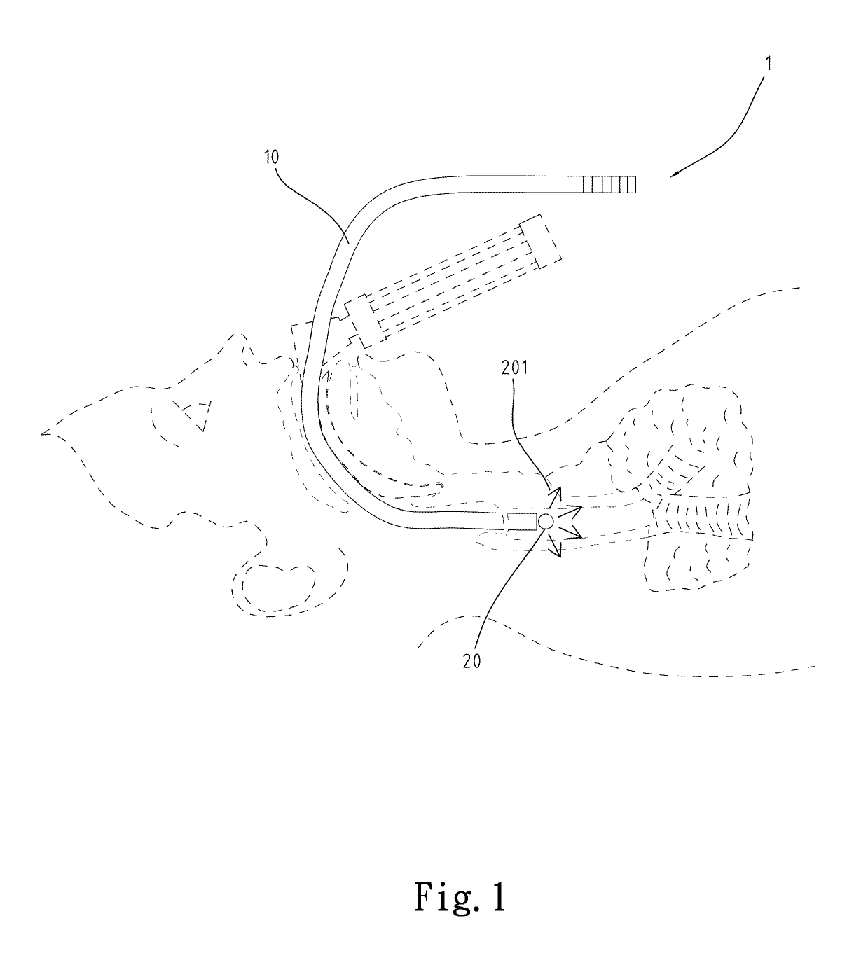

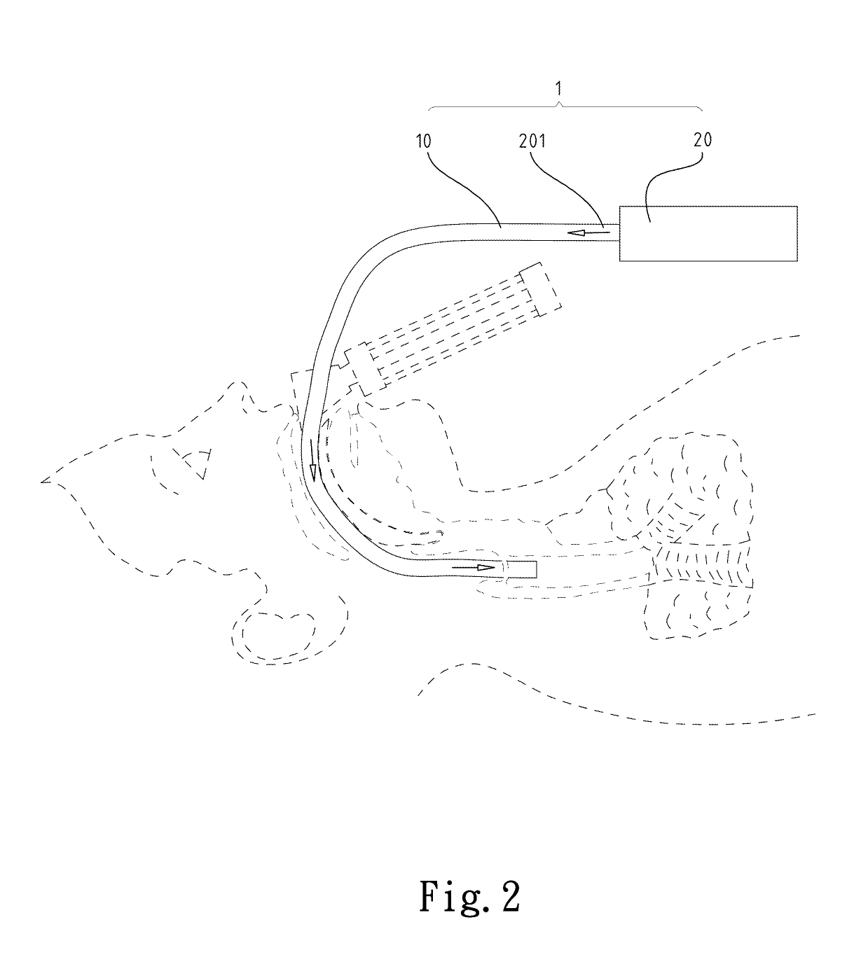

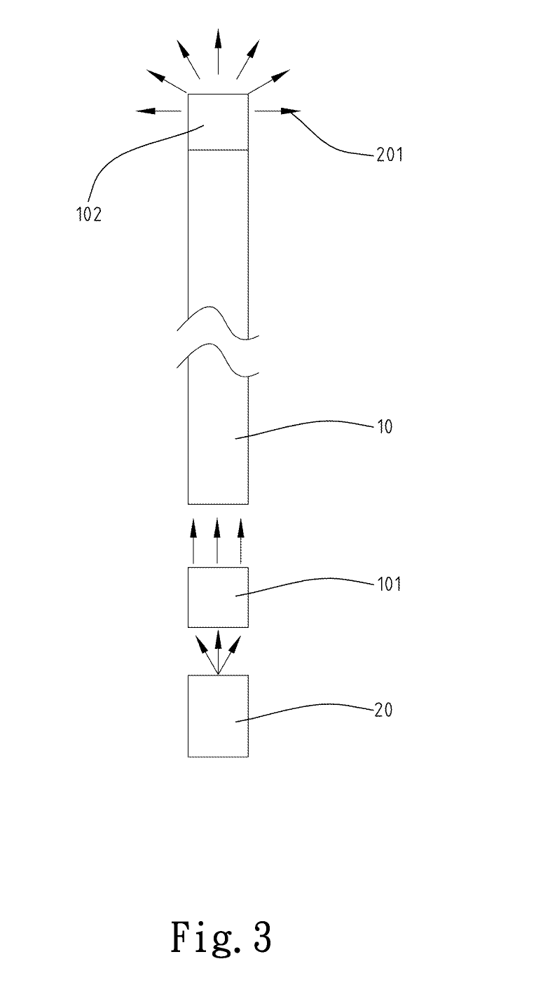

FIG. 1 is a schematic view of a light guide for assisting intubation according to a first embodiment of the invention. FIG. 2 is a schematic view of a light guide for assisting intubation according to a second embodiment of the invention. FIG. 3 is a schematic view of a light receiving element and a light scattering element of a light guide for assisting intubation according to the second embodiment of the invention.

As shown, a light guide 1 for assisting intubation includes a catheter 10 and a light-emitting element 20.

The catheter 10 is made of material with plasticity. In practical applications the material can be changed based on the actual conditions. The light emitting element 20 is mounted in a detachable manner at one end of the catheter 10 for emitting a light 201. Detachable design is well known in the art, and can be realized without any detailed description in this specification. For example, a plug or a clip matching the diameter of the light emitting element 20 can be directly interposed or sandwiched to the light emitting element 20, which is easily accomplished for those who are skilled in the art.

In a preferred embodiment, the light emitting element 20 may be a light source having directivity, for example: a commercially available light emitting diode. When the end of the light emitting element 20 of the catheter 10 is inserted into an animal's body (a human body is exemplified for illustration, it is intended to limit the scope of the invention to this example). The light 201 from the light-emitting element 20 penetrates the body surface of the animal in order to indicate the correct position where the catheter 10 is inside the animal's body. It is noted that in order to facilitate the light 201 to penetrate through the body surface of the animal (particularly referring to a human body), the light 201 can be a visible light of wavelength from 590 nm to 760 nm. In other words, it can be a light emitting diode which emits a red light.

Furthermore, in another embodiment of the present invention, the light emitting element 20 is mounted at one end of the catheter 10 which does not enter the human body. Taking advantage of the plasticity and light transmissibility of the material for the catheter 10, for example, for a fiber catheter, the light 201 form the light emitting element 20 is efficiently transmitted to the front end. Thereby, the light 201 can be transmitted through the animal's body.

Referring to FIG. 3, in order to allows the light 201 to be efficiently scattered from the interior of the animal to the surface of the animal body, a light receiving element 101 and a light scattering element 102 are respectively mounted at either ends of the catheter 10, and the light emitting element 20 is mounted adjacent to the light receiving element 101. In other words, the light 201 from the light emitting element 20 can be moderately aggregated through the light receiving element 101, and then go into the catheter 10 optionally through the above fiber. When the light 201 travels to the other end of the catheter 10, the light 201 effectively scatters through the role of the light scattering element 102, resulting in more efficient penetration through the animal's neck skin. The light receiving element, also known as a light condenser, can be made by the concave lens or a few refractors. It has several types. Similarly, the light scattering element can be also composed by a convex lens or a few slices of refractors. Those who are skilled in the art can fully understand without further description.

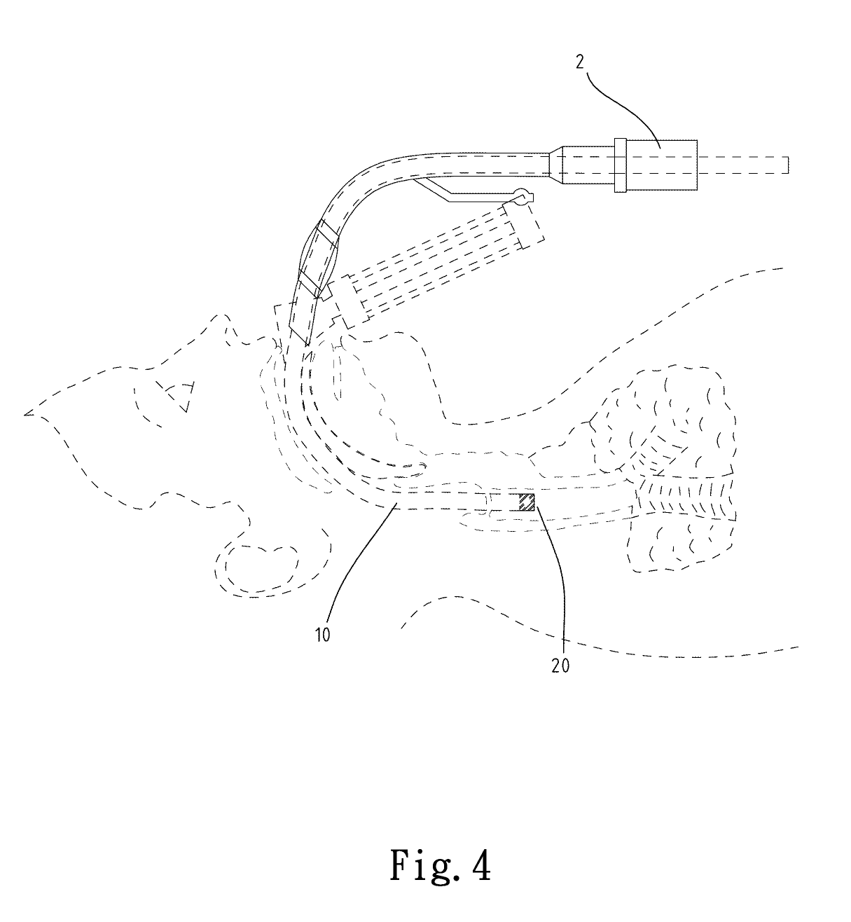

FIG. 4 is a schematic view of intubation assisting by means of the light guide according to one embodiment of the invention. As shown, in the actual medical intubation emergency operation, the medical personnel needs to push away the patient's tongue and then place a laryngoscope blade at the junction of the base of the tongue and the epiglottis so that the physician can see the vocal cord then puts the endotracheal tube into vocal cord. Such an operation is not only time consuming but also easy to occur medical errors caused by wrong-positioned intubation due to the patient's tamper when the patient feels suffering. For this reason, with the use of the light guide 1 for assisting intubation according to the present invention, the intubation operation can be quickly and accurately completed.

As shown, the light guide 1 for assisting intubation can cooperate with an intubation apparatus 2. When the medical personnel is about to do intubation operation to a patient, he or she can use the light guide 1 of the present invention to confirm the position where the intubation apparatus 2 is to be. After the position of the catheter is confirmed, the intubation apparatus 2 is sleeved to the catheter 10 for the intubation apparatus 2 endotracheal tube directly slides into the human's vocal cord along the catheter 10. After the intubation apparatus 2 reaches the positions marked by light, the intubation operation is completed. When the intubation operation is completed, the catheter 10 can is directly pulled out of the intubation apparatus 2.

Specifically, with the assistance of the red light (light 201) emitting by the light emitting element 20 and transmitting out of the body, the medical personnel first place the catheter 10 into the body in the patient's mouth or nasal passages and reach the right location. After the intubation position is confirmed, the intubation apparatus 2 is sleeved to the catheter 10 and reach the patient's trachea along the catheter 10. Then, the catheter 10 is unplugged to complete the intubation operation.

It is noted that, in the practical application, if the medical personnel cannot see the patient's glottis in the intubation process, the front end of the catheter 10 can be bent to a certain curve for trying to go further along the patient's epiglottis by anatomy. Therefore, the location where the light 201 emitted by the light emitting element 20 of the catheter 10 can help to judge whether the catheter 10 has reached the position of the trachea or not. In other words, after the laryngoscope to provoke the airway, if the epiglottis covers the glottis, the catheter 10 can be used to poke the epiglottis. Thereby, the medical staff can see the glottis, and accordingly determines which direction the catheter 10 should advance to complete the positioning of the catheter 10.

In summary, the light guide for assisting intubation according to the present invention, the medical personnel can clearly see the light of the light emitting element at the position of the Adam's apple, and thereby confirm the catheter is indeed in the trachea. It can avoid any mistakes in determining the completion of intubation because the patient's breath sounds cannot be clearly recognized by the stethoscope. It can also avoid the risk of hypoxia happening in waiting for figuring out the result of chest X-ray examination. That is, the light guide of the present invention has improved the inconvenience using the stethoscope or chest X-ray to determine the position of the catheter in the endotracheal, saving of intubation operation for medical personnel.

The descriptions illustrated supra set forth simply the preferred embodiments of the present invention; however, the characteristics of the present invention are by no means restricted thereto. All changes, alternations, or modifications conveniently considered by those skilled in the art are deemed to be encompassed within the scope of the present invention delineated by the following claims.

What is claimed is:

1. A light guide for assisting intubation, comprising:
a catheter, made of material having plasticity; and
a light emitting element, emitting a light which can penetrate through the animal's body and mounted at one end of the catheter;
wherein when the end of the catheter having the light emitting element inserts in the animal's body, the light from the light emitting element penetrates through animal's body to indicate the location of that end of the catheter.

2. The light guide of claim 1, wherein the light emitting element is a light source having directivity.

3. The light guide of claim 1, wherein the light emitting element is a light emitting diode.

4. The light guide of claim 1, wherein the light from the light emitting element is the visible light having wavelength of 590 nm-760 nm.

5. The light guide of claim 1, wherein the light emitting element is mounted in a detachable manner.

6. A light guide for assisting intubation, comprising:
a catheter, made of material having plasticity and light transmissibility; and
a light emitting element, emitting a light which can penetrate through the animal's body and mounted at one end of the catheter;
wherein when the end of the catheter having no light emitting element inserts in the animal's body, the light from the light emitting element travels through the catheter, goes out of other end of the catheter and then penetrates through the animal's body to indicate the location of that end of the catheter.

7. The light guide of claim 6, wherein the catheter is made of optical fiber.

8. The light guide of claim 6, wherein the end of the catheter where the light emitting element is provided with has a light receiving element to receive the light from the light emitting element for guiding the light into the catheter.

9. The light guide of claim 6, wherein the end of the catheter where the light emitting element is not provided with has a light scattering element to scatter the light.

10. The light guide of claim 6, wherein the light emitting element is a light source having directivity.
